(19) United States
(12) Patent Application Publication
(10) Pub. No.: US 2012/0259116 A1
(43) Pub. Date: Oct. 11, 2012

(54) Novel Process for the Preparation of Paliperidone

(76) Inventors: Rajiv Kumar, Kharghar (IN); Dharmesh Kumar Arvindbhaipatel, Kharghar (IN); Dattatray Shamrao Metil, NaviMumbai (IN); Praveen Raosaheb Supekar, Amhednagar (IN); Prashant Pandurangpawar, NaviMumbai (IN); Santosh Vitthal Pune, Kalyan (IN)

(21) Appl. No.: 13/516,795

(22) PCT Filed: Dec. 20, 2010
(86) PCT No.: PCT/IN2010/000830

§ 371 Date: Jun. 18, 2012

(30) Foreign Application Priority Data
Dec. 17, 2009 (IN) 2916/MUM/2009

(51) Int. Cl. C07D 471/04

(52) U.S. Cl. 544/282

(57) ABSTRACT

The present invention relates to a novel process for the preparation of paliperidone by hydrolysis of 9-O-acylated paliperidone. In a preferred embodiment of the present invention, Paliperidone Form (II) of purity of about 98% or more was obtained by basic hydrolysis of 9-O-Acetyl Paliperidone.

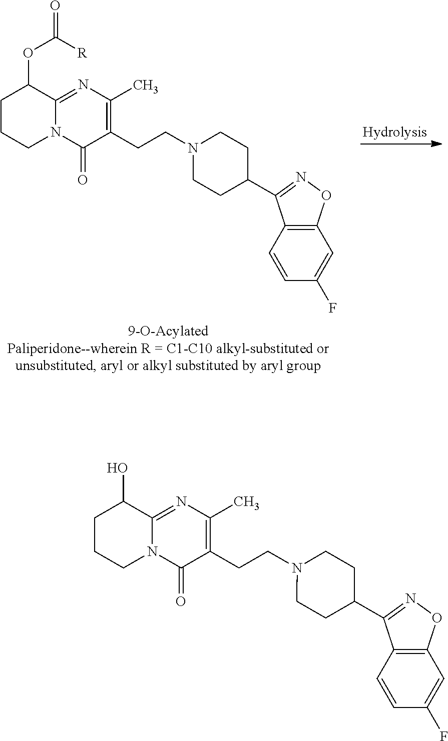

### FIELD OF THE INVENTION

The present invention relates to a novel process for the preparation of paliperidone.

### BACKGROUND OF THE INVENTION

Paliperidone, 3-[2-[4-(6-fluorobenzo[d]isoxazol-3-yl)-1-piperidyl]ethyl]-7- hydroxy-4-methyl- 1,5-diazabicyclo[4.4.0]deca-3,5-dien-2-one, is a 5-HT antagonist belonging to the chemical class of benzisoxazole derivatives and has a racemic mixture of the following structural Formula:

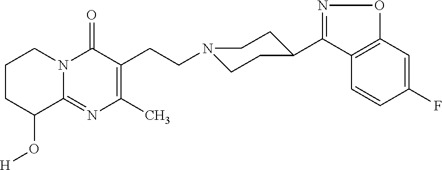

Paliperidone, the primary active metabolite of the existing antipsychotic risperidone is 9-hydroxyrisperidone, i.e., risperidone with an extra hydroxyl group. Paliperidone (Invega), known as second generation antipsychotic drug developed by Janssen Pharmaceuticals is an extended release formulation of paliperidone that employs an oral osmotic extended release delivery system for once- daily dosing. Paliperidone was approved by FDA for the treatment of schizophrenia on Dec. 20, 2006. It was initially marketed for the treatment of schizophrenia and then for bipolar mania.

Processes for the synthesis of paliperidone are described in U.S. Pat. No. 5,158,952, U.S. Pat. No. 5,158,952 (herein referred to as US '952) discloses paliperidone and a process for the preparation of paliperidone, wherein 3-(2-chloroethyl)-2-methyl-9-(phenylmethoxy)-4H-pyrido[1,2-a]pyrimidin-4-one is hydrogenated to obtain 3-(2-chloroethyl)-6,7,8,9-tetrahydro-9-hydroxy-4H- pyrido[1,2-a]pyrimidin-4-one which is subsequently condensed with 6- fluoro-3-(4-piperidinyl)- 1,2-benzisoxazole mono hydrochloride in presence of a base in a solvent, preferably methanol to obtain paliperidone. Following alkylation reaction, methanol is removed under reduced pressure to give oily residue followed by dissolving the residue in chloroform and washing with water.

The organic layer is dried, concentrated under reduced pressure to obtain residue, subjected to column chromatographic purification using a mixture of chloroform and methanol, followed by evaporation of eluants to give residue. The residue so obtained was subjected to repeated crystallizations in different solvents like acetone and isopropyl alcohol to give paliperidone having the melting point 179.8° C.

Scheme-1 illustrates the process disclosed in U.S. Pat. No. 5,158,952for the synthesis of Paliperidone.

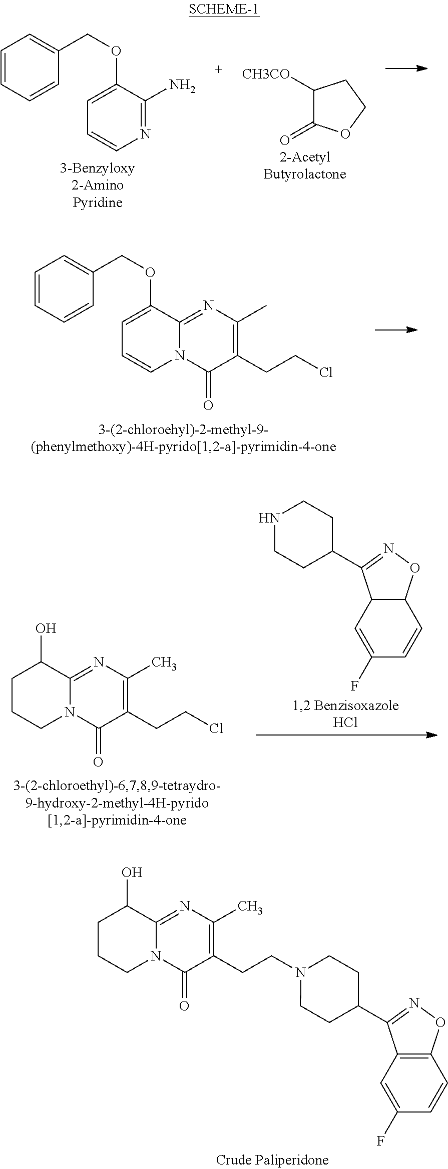

The major drawbacks of the US '952 process are:
US '952 employs halogenated solvents of the like of Chloroform combined with MeOH and NH3 as eluants for Column Chromatography for the purification of the Crude Paliperidone obtained.
Additionally the Column purified Paliperidone was subjected to crystallization with 2-propanone; followed by 2-propanol: Diethyl ether wash & finally Re-crystallisation from 2-propanol.
US '952 does not provide the Purity of the Paliperidone so obtained even after repeated purification techniques from a number of solvents including Column chromatographic purification.
Catalytic hydrogenation stage with Pd—C resulted in incomplete reaction, vigorous reaction conditions, long time for completion and uncontrolled side products.
Also the overall yield of the Final Paliperidone is low.

Thus the US '952 process not only employs Column Chromatography for Purification with solvents, it also necessitates the use of Recrystallisation techniques with solvents in its attempt to purify Paliperidone. Column chromatography technique for purification is very cumbersome and thus not suitable on industrial scale operations. Also solvents of the like of Chloroform are hazardous and injurious to health and are not environment friendly.

U.S. Pat. No. 5,254,556 discloses a process for preparing C₂₋₂₀ alkanoic acid ester and their pharmaceutically acceptable acid addition salts. But it does not suggest a process for purifying Paliperidone by the Hydrolysis of these esters.

PCT publication WO 2008/021342, discloses the various crystalline forms I, II, III, IV, V, VI and also Amorphous Forms of paliperidone. Recrystallization of the crude Paliperidone from various solvents produced various polymorphic forms of paliperidone. WO 2008/021342 also discloses that pure crystalline Form II of paliperidone can be obtained by crystallization from a solution of paliperidone and a solvent. However, purity of the paliperidone is not disclosed in this patent application.

US20090048272 discloses processes for preparing paliperidone via a novel intermediate 3-(2- chloroethyl)-6, 7, 8, 9-tetrahydro-9-acetoxy-2-methyl-4H-pyrido-[1,2-a]-pyrimidrn-4-one and condensing cither this or its Acetyl deprotected free 9-Hydroxy derivative with 1,2-Benzisoxazole HCl to afford the Crude paliperidone which was further purified by recrystallisation.

WO2009060297A2 teaches an improved process for the preparation of paliperidone by making its acid addition salts.

As discussed above none of the methods taught by the prior arts were successful neither in their attempt to obtain Paliperidone of high purity or provide an industrially viable, economic or eco-friendly process for Paliperidone Form II. Impurities in any API including Paliperidone are totally undesirable and hence there is an imperative need to provide Paliperidone of high purity devoid of extraneous impurities that can come from any sources.

When the inventors of the present invention attempted to purify the crude paliperidone by prior art teachings such as by crystallization from solvents, this posed technical problems in that it necessitated large amounts of solvent due to low solubility of the crude Paliperidone possessing considerably large amount of impurities. This propelled the inventors of the present invention to find out alternative purification techniques that would result in Paliperidone of superior quality and also facilitate industrial feasibility of the process.

Surprisingly, the present inventors discovered that the Crude paliperidone can be easily converted to its 9-O-Acylated Paliperidone derivative. The 9-O-Acylated Paliperidone derivative in turn afforded easy purification by the conventional recrystallisation processes resulting in 9-O-Acylated Paliperidone of high quality. Surprisingly the present inventors discovered that this 9-O-Acylated Paliperidone derivative when subjected to hydrolysis directly afforded Paliperidone Form II in high yield and purity. Also the Paliperidone thus obtained did not require any further purification processes and can be directly forwarded to the Formulation stage.

### OBJECTIVE OF THE INVENTION

The main objective of the invention is to provide Paliperidone Form II suitable for Pharmaceutical purposes in high purity and high yield.

Another objective of the invention is to provide a simple, industrially amenable and eco-friendly purification process for crude paliperidone without the use of cumbersome Column chromatography or repeated Recrystallisation techniques.

### SUMMARY OF THE INVENTION

In view of the difficulties encountered by the prior art processes in the commercial preparation of pure paliperidone, we aimed to develop a simple and economically viable process for the commercial production of pure paliperidone or form II directly by hydrolysis of 9-O-Acylated Paliperidone.

Technical or Crude paliperidone may be synthesized by known prior art techniques or as per the process described in the prior art U.S. Pat. No. 5,158,952.

An embodiment of the present invention is illustrated in Scheme-2 & Scheme-3.

Scheme-2 illustrates an embodiment of the present invention comprising conversion of Crude Paliperidone to its 9-O-Acyl derivative which is purified by standard recrystallisation techniques. This on hydrolysis with a Methanolic Ammonia or Methyl amine afforded Paliperidone with a purity of about 98.0% or more.

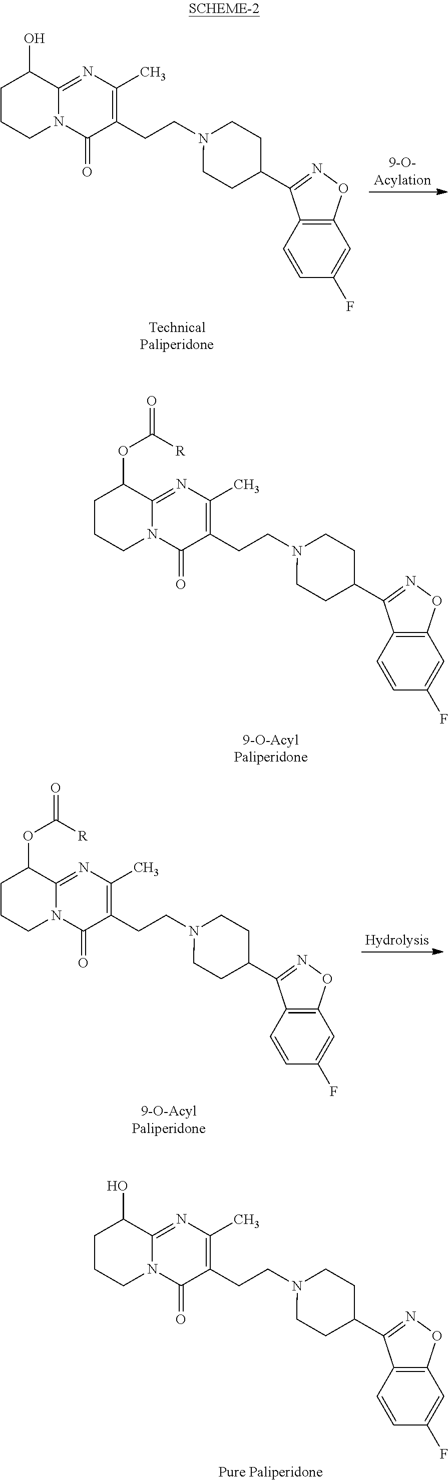

Scheme-3 illustrates another embodiment of the present invention comprising conversion of Crude Paliperidone to its 9-O-Acetyl derivative which is purified by standard recrystallisation techniques. This on hydrolysis with a Methanolic Ammonia or Methyl amine afforded the desired paliperidone Form-II with a purity of about 98.0% or more.

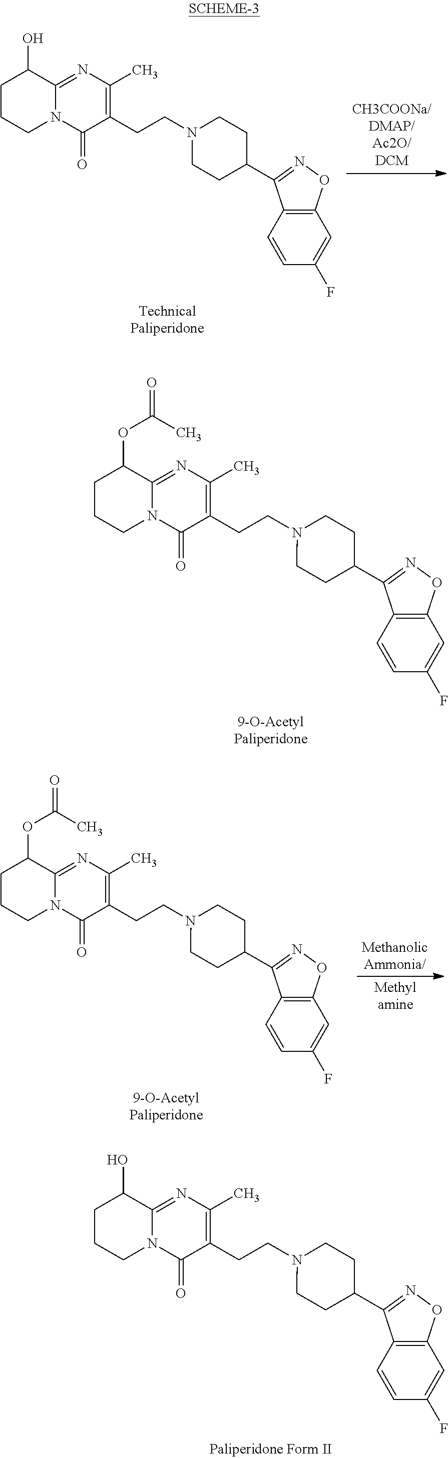

The process of the present invention may be summarized as follows:
A. A process for the preparation of paliperidone comprising hydrolysis of 9-O-acylated Paliperidone of Formula

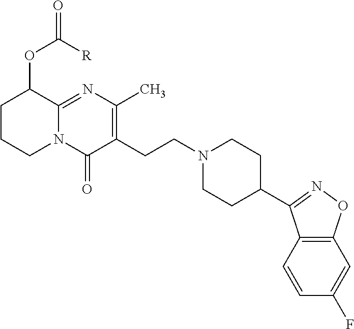

wherein R=C₁₋C₁₀ straight or branched alkyl, alkyl substituted by aryl or aryl which may be unsubstituted or substituted.
B. The process according to A wherein the Paliperidone obtained is form II.
C. The process according to A where the Paliperidone has a purity of about 98% or more.
D. The process according to A comprising hydrolysis of 9-O acetyl paliperidone of Formula

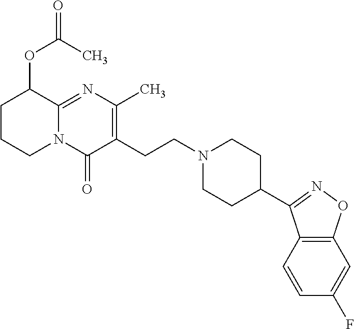

to afford Paliperidone Form II of purity of about 98% or more.
E. The process according to A wherein the hydrolysis is acidic or basic.
F. The process according to E wherein the base used for hydrolysis is selected from inorganic or organic bases selected from the group comprising sodium carbonate, potassium carbonate, sodium hydroxide, potassium hydroxide, ammonia, sodium methoxide, sodium ethoxide, potassium t-butoxide, triethyl amine, diisopropylamine, methyl amine and the like.
G. The process according to F where the base is preferably selected from Ammonia or Methylamine.
H. The process according to A comprising
   a) Conversion of crude Paliperidone to 9-O-acylated paliperidone and optionally purifying it.
   b) Hydrolysis of 9-O-Acylated paliperidone.

### BRIEF DESCRIPTION OF DRAWING FIGURES

Further objects of the present invention together with additional features contributing thereto and advantages accruing there from will be apparent from the following description of preferred embodiments of the invention which are shown in the accompanying drawing figures wherein:

Scheme-1 illustrates the process disclosed in U.S. Pat. No. 5,158,952 for the synthesis of Paliperidone.

Schemes-2 & 3 illustrate embodiments of the present invention.

FIG. 1 provides the X-ray powder diffraction of Paliperidone Form II obtained by the present invention.

### DETAILED DESCRIPTION OF THE INVENTION

Before the present process and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention relates to a novel process for the preparation of paliperidone from 9 O acylated Paliperidone.

According, to another aspect of the present invention is provided a process for the preparation/purification of Paliperidone, the process comprising subjecting 9 O acylated Paliperidone to hydrolysis to afford paliperidone directly with a purity of about 98% or more, preferably about 99% or more without the use, of conventional column purification techniques or repeated recrystallisations from solvents.

Technical or Crude paliperidone may be synthesized by known prior art techniques or as per the process described in the prior art U.S. Pat. No. 5,158,952.

Crude paliperidone may be converted to 9 O acylated Paliperidone by known prior art techniques or by the US '952 procedure.

Thus according to an aspect of the present invention, is provided a process for the preparation/purification of paliperidone by its conversion to 9 O acylated Paliperidone.

Crude Paliperidone may be converted to any suitable 9-O-acylated derivative of formula

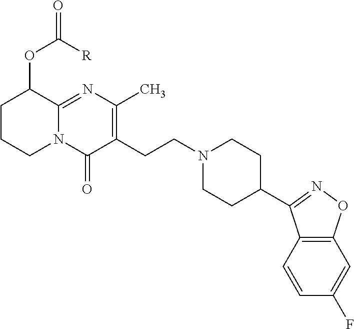

wherein R=C₁-C₁₀ straight or branched alkyl, alkyl substituted by aryl or aryl which may be substituted or unsubstituted.

In embodiments of the present invention, the 9-O-acylated Paliperidone may be selected from a group comprising 9-O-propionyl Paliperidone, 9-O -Phenylacetyl Paliperidone, 9-O-benzoyl Paliperidone and the like. The 9-O-acylated paliperidone may optionally be purified by conventional techniques and then subject to hydrolysis in a suitable solvent in the presence of an acid or a base. The Paliperidone so obtained may optionally be converted to other Polymorphic forms.

The mode of hydrolysis of 9-O-Acylated Paliperidone may be acidic or basic or any other conventional method available in the prior art. The acids for hydrolysis may be selected from the group of inorganic or organic acids selected from a group comprising hydrochloric acid, hydrobromic acid, sulfuric acid, p-toluenesulfonic acid, methanesulfonic acid, acetic acid, trifluoroacetic acid, and the like or mixtures thereof. The bases may be selected from a group of inorganic and organic bases selected from a group comprising sodium carbonate, potassium carbonate, sodium hydroxide, potassium hydroxide, ammonia, sodium methoxide, sodium ethoxide, potassium t-butoxide, triethyl amine, diisopropylamine, Monomethylamine and the like or mixtures thereof; preferably Methylamine or Ammonia or the like.

The reaction may be carried out at any temperature between ambient temperature and the boiling point of the solvent. The term ambient temperature in the present application is intended to indicate the temperature usually found in industrial laboratory and production facilities such as in the range of 15-30° C., preferably in the range of 20-25° C.

The hydrolytic cleavage reaction may be conducted in a suitable solvent such as water, alcohol, ester, ketone, nitrile, hydrocarbon, ether and the like or mixtures thereof. Preferred solvents are water, alcohols, ethers and mixtures thereof.

In an embodiment of the present invention, crude paliperidone was reacted with acetic anhydride in the presence of DMAP/Na acetate in DCM under reflux until absence of starting material. DCM was removed. Water was added followed by aq.NH3 and the pH adjusted to about 6.5 to 9.0. The solid obtained was filtered and dried under vacuum at 50-55 C to afford 9-O-Acetyl Paliperidone. 9-O-Acctyl Paliperidone was purified by recrystallisation by standard procedures known to a person skilled in art.

In an embodiment of the present invention, 9-O-acetyl Paliperidone was dissolved in a suitable solvent, charcoalised, filtered. The clear solution then cooled to afford the Pure 9-O-acetyl Pailiperidone.

The solvents for recrystallisation may be any suitable solvent such as water, alcohol, ester, ketone, nitrile, hydrocarbon, ether and the like or mixtures thereof.

In embodiments of the present invention, 9-O-acetyl paliperidone was purified by recrystallisation from solvents such as Acetone, Ethanol, Ethyl acetate, Toluene and the like. In an embodiment of the present invention, 9-O-Acetyl Paliperidone was subjected to hydrolysis in a solvent at ambient temperature. The precipitated solid was filtered, washed with solvent and dried under vacuum to afford Paliperidone form II. Paliperidone form II with purity more than 99% was thus obtained directly without the use of any conventional solvent recrystallisation techniques or cumbersome column purification techniques. The Paliperidone so obtained was directly forwarded for Formulation stage without any additional purification steps.

The following examples are intended to illustrate the scope of the present invention in all its aspects but not to limit it thereto.

### EXAMPLES

Although the invention has been described in terms of particular embodiments and applications, one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are preferred by way of example to facilitate comprehension of the invention and should not be construed to limit the scope thereof.

### Example-1

### Preparation of 9-O-Acetyl Paliperidone from Crude Paliperidone

Acetic anhydride (123 g, 1.2048 moles) was added slowly in about 20 minutes time to a mixture of Crude paliperidone (315 g, 0.74 moles), sodium acetate (121 gm, 1.4770 moles), DMAP (3.1 g, 0.025 moles) in DCM (3150 ml) and the reaction mass maintained at reflux for 4 hrs until absence of starting material. DCM was removed. Water was added followed by aq.NH3 and the pH adjusted to 6.5-7.0. The solid obtained was filtered and dried under vacuum at 50-55° C. Dry Wt—325 g.

### Example-2

### Purification of 9-O-Acetyl Paliperidone

A clear, solution of the crude 9-O-Acetyl Paliperidone (180 g, 0.385 moles) in acetone (1620 ml, 9 v/w) at 50-55 C was treated with Charcoal for 15 minutes. The RM was then filtered under hyflo bed and cooled slowly to about 15-20 C and stirred at this temp fop 1 hr. The precipitated solid was then filtered, washed with acetone and dried under vacuum at 45 C Dry Wt—135 g.

### Example-3

### Preparation of Paliperidone FORM II

Methanolic ammonia (3740 ml, 17 v/w) was added slowly to 9-O-Acetyl Paliperidone (220 g, 0.47 moles) and the RM slowly heated at 40 C for 6 hrs. The RM was cooled to 30 C and Methanolic ammonia (440 ml, 2 v/w) was further added and the RM stirred at RT overnight. The precipitated solid was filtered, washed with methanol, and dried under vacuum at 45 C for 12 hrs. Dry Wt—186 g. Purity=99.05%

### Example-4

### Preparation of Paliperidone FORM II

Methanolic ammonia (2720 ml, 17 v/w) was added slowly to 9-O-Acetyl Paliperidone (160 g 0.3415 moles) and the RM slowly heated at 40° C. for 6 hrs. The RM was cooled to 30° C. and Methanolic ammonia (320 ml, 2 v/w) was further added and the RM stirred at RT overnight. The precipitated solid was filtered, washed with methanol, and dried under vacuum at 45° C. for 12 hrs. Dry Wt—136 g. Purity=99.05%

### Example-5

### Preparation of Paliperidone FORM II

Methanolic ammonia (1200 ml, 16 v/w) was added slowly to 9-O-Acetyl Paliperidone (75 g, 0.16 moles) and the RM slowly heated at 40° C. for 6 hrs. The RM was cooled to 30° C. and Methanolic ammonia (150 ml, 2 v/w) was further added and the RM stirred at RT overnight. The precipitated solid was filtered, washed with methanol, and dried under vacuum at 45° C. for 12 hrs. Dry Wt—65 g. Purity=99.05%

### Example-6

### Preparation of 9-O-Acetyl paliperidone from Crude Paliperidone

Acetic anhydride (229.80 gm, 2.2509 moles) was added slowly in about 20 minutes time to a mixture of Crude paliperidone (600 gm, 1.41 moles), DMAP (6 gm, 0.050 moles), sodium acetate (231 gm, 2.8136 moles) and methylene chloride (3000 ml) and reaction mass maintained at reflux for 4 hrs until absence of starting material: Cooled reaction mass to 25-30° C. Charged DM water (3000 ml) followed by aq.NH₃and pH adjusted to 7.5 to 9.0. Separate the organic layer, charge activated carbon (30 gm). Filter the organic layer and distilled out under vacuum at 35-40° C., strip out with Acetone (600 ml), charge Acetone (2400 ml) heated to 50-55° C., stirred for 30 min, cooled to 0-5° C., stirred for 2 hr at 0-5° C. The solid was filtered washed with chilled acetone (1200 ml) and dried in oven at 45-50° C. for 12 hrs. Dry Wt:- 510 g.

### Example-7

### Preparation of 9-O-Acetyl paliperidone from Crude Paliperidone

Acetic anhydride (4.0 gm, 0.0391 moles) was added slowly in about 20 minutes time to a mixture of Crude paliperidone (10 gm, 0.023 moles), DMAP (0.1 gm, 0.0008 moles), sodium acetate (3.8 gm, 0.046 moles) and Acetone (50 ml) and reaction mass maintained at reflux for 4 hrs until absence of starting material. Cooled reactions mass to 25-30° C. Filter the inorganic solid. Charged aq.NH3 and pH adjusted to 7.5 to 9.0. Cooled reaction mass to 5-10° C. Stirred for 2 hr at 5-10° C. The solid was filtered and dried in oven at 45-50° C. for 12 hrs. Dry Wt:- 6.5 g.

### Example-8

### Purification of 9-O-Acetyl Paliperidone

A clear solution of the crude 9-O-Acetyl Paliperidone (40 g, 0.085 moles) in ethanol (520 ml,) at 50-55° C. was treated with Charcoal for 15 minutes. The RM was then filtered under hyflo bed and cooled slowly to about 15-20° C. and stirred at this temp for 1 hr. The precipitated solid was then filtered, washed with ethanol (50 ml) and dried under vacuum at 45° C. Dry Wt—28 g.

### Example-9

### Purification of 9-O-Acetyl Paliperidone

A clear solution of the crude 9-O-Acetyl Paliperidone (40 g, 0.085 moles) in Toluene (240 ml) at 50-55° C. was treated with Charcoal for 15 minutes. The RM was then filtered under hyflo bed and cooled slowly to about 15-20° C. and stirred at this temp for 1 hr. The precipitated solid was then filtered, washed with toluene (50 ml) and dried under vacuum at 45° C. Dry Wt—20 g.

### Example-10

### Purification of 9-O-Acetyl Paliperidone

A clear solution of the crude 9*6-Acetyl Paliperidone (40 g, 0.085 moles) in ethyl acetate (360 ml,) at 50-55° C. was treated with Charcoal for 15 minutes. The RM was then filtered under hyflo bed and cooled slowly to about 15-20° C. and stirred at this temp for 1 hr. The precipitated solid was then filtered, washed with ethyl acetate (50 ml) and dried under vacuum at 45° C. Dry Wt—23 g.

### Example-11

### Purification of 9-O-Acetyl Paliperidone

A clear solution of the crude 9-O-Acetyl Paliperidone (40 g, 0.085 moles) in methanol (200 ml,) at 50-55° C. was treated with Charcoal for 15 minutes. The RM was then filtered under hyflo bed and cooled slowly to about 15-20° C. and stirred at this temp for 1 hr. The precipitated solid was then filtered, washed with methanol (50 ml) and dried under vacuum at 45° C. Dry Wt—30 g.

### Example-12

### Purification of 9-O-Acetyl Paliperidone

A clear solution of the crude 9-O-Acetyl Paliperidone (40 g, 0.085 moles) in acetonitrile (360 ml,) at 50-55° C. was treated with Charcoal for 15 minutes. The RM was then filtered under hyflo bed and cooled slowly to about 15-20° C. and stirred at this temp for 1 hr. The precipitated solid was then filtered, washed with acetonitrile (50 ml) and dried under vacuum at 45° C. Dry Wt—25 g.

### Example-13

### Preparation of Paliperidone FORM-II

Methanolic monomethyl amine (25%) (1200 ml, 8 v/w) was added slowly to 9-O-Acetyl Paliperidone (150 gm, 0.32 moles) in Methylene chloride (1500 ml,) at 25-30° C. Stirred reaction mass for 8 hr at 25-30° C., Distilled out solvent under vacuum at 35-40° C., up 5 v remain inside the flask. The precipitated solid cooled to 25-30° C. stirred for 2 hr then filtered, washed with methanol (300 ml), and dried under vacuum at 45-50° C. for 12 hrs. Dry Wt:- 120 g. Purity=99.75%

### Example-14

### Preparation of Paliperidone FORM-II

Monomethyl amine (40%) (2000 ml, 8 v/w) was added slowly to 9-O-Acetyl Paliperidone (250 gm, 0.5336 moles) in* methanol (3750 ml,) at 30-35° C. Stirred reaction mass for 8 hr at 30-35° C., then RM was cooled to 30° C. The precipitated solid was filtered, washed with methanol (500 ml), and dried under vacuum at 45-50° C. for 12 hrs. Dry Wt:- 200 g. Purity=99.75%

### Example-15

### Preparation of 9-O-Acetyl paliperidone from Crude Paliperidone

Acetyl chloride (13.80 gm, 0.1758 moles) was added slowly at 0-5° C. in about 40 minutes time to a mixture of Crude paliperidone (50 gm, 0.1172 moles), Triethyl amine (23.4 4 gm, 0.2344 moles) and methylene chloride (400 ml) and reaction mass maintained at 10-15° C. for 4 hrs until absence of starting material. Charged DM water (400 ml), stirred for 15 min. separate the organic layer, charge activated carbon (30 gm). Filter the organic layer and distilled out under vacuum at 35-40° C., strip out with Acetone (50 ml), charge Acetone (150 ml) heated to 50-55° C., stirred for 30 min, cooled to 0-5° C., stirred for 2 hr at 0-5° C. The solid was filtered washed with chilled acetone (50 ml) and dried in oven at 45-50° C. for 12 hrs.

Dry Wt:- 40 g. Crude product was re-crystallized from acetone Dry Wt:- 35 g.

### Example-16

### Preparation of 9-O-Propionyl paliperidone from Crude Paliperidone

Propionyl chloride (16.27 gm, 0.1758 moles) was added slowly at 0-5° C. in about 40 minutes time to a mixture of Crude paliperidone (50 gm, 0.1172 moles), Triethyl amine (23.4 4 gm, 0.2344 moles) and methylene chloride (400 ml) and reaction mass maintained at 10-15° C. for 4 hrs until absence of starting material. Charged DM water (400 ml), stirred for 15 min. separate the organic layer, charge activated carbon (30 gm). Filter the organic layer and distilled out under vacuum at 35-40° C., strip out with Acetone (50 ml), charge Acetone (150 ml) heated to 50-55° C., stirred for 30 min, cooled to 0-5° C., stirred for 2 hr at 0-5° C. The solid was filtered washed with chilled acetone (50 ml) and dried in oven at 45-50° C. for 12 hrs. Dry Wt:- 41 g. Crude product was recrystallized from acetone. Dry Wt:- 35 g.

### Example-17

### Preparation of 9-O-Benzoyl Paliperidone from Crude Paliperidone

Benzoyl chloride (24.71 gm, 0.1758 moles) was added slowly at 0-5° C. in about 40 minutes time to a mixture of Crude paliperidone (50 gm, 0.1172 moles), Triethyl amine (23.4 4 gm, 0.2344 moles) and methylene chloride (30.0 ml) and reaction mass maintained at 10-15° C. for 4 hrs until absence of starting material. Charged DM water (300 ml), stirred for 15 min. separate the organic layer, charge activated carbon (30 gm). Filter the organic layer and distilled out under vacuum at 35-40° C., strip out with Acetone (50 ml), charge Acetone (150 ml) heated to 50-55° C., stirred for 30 min, cooled to 0-5° C., stirred for 2 hr at 0-5° C. The solid was filtered washed with chilled acetone (50 ml) and dried in oven at 45-50° C. for 12 hrs. Dry Wt:- 40 g. Crude product was re-crystallized from acetone. Dry Wt:- 35 g.

### Example-18

### Preparation of 9-O-Phenylacetyl Paliperidone from Crude Paliperidone

Phenyl acetyl chloride (27.17 gm, 0.1758 moles) was added slowly at 0-5° C. in about 40 minutes time to a mixture of Crude paliperidone (50 gm, 0.1172 moles), Triethyl amine (23.4 4 gm, 0.2344 moles) and methylene chloride (300 ml) and reaction mass maintained at 10-15° C. for 4 hrs until absence of starting material. Charged DM water (300 ml), stirred for 15 min. separate the organic layer, charge activated carbon (30 gm). Filter the organic layer and distilled out under vacuum at 35-40° C., strip out with Acetone (50 ml), charge Acetone (150 ml) heated to 50-55° C., stirred for 30 min, cooled to 0-5° C., stirred for 2 hr at 0-5° C. The solid was filtered washed with chilled acetone (50 ml) and dried in oven at 45-50° C. for 12 hrs. Dry Wt:- 44 g. Crude product was re-crystallized from acetone. Dry Wt:- 37 g.

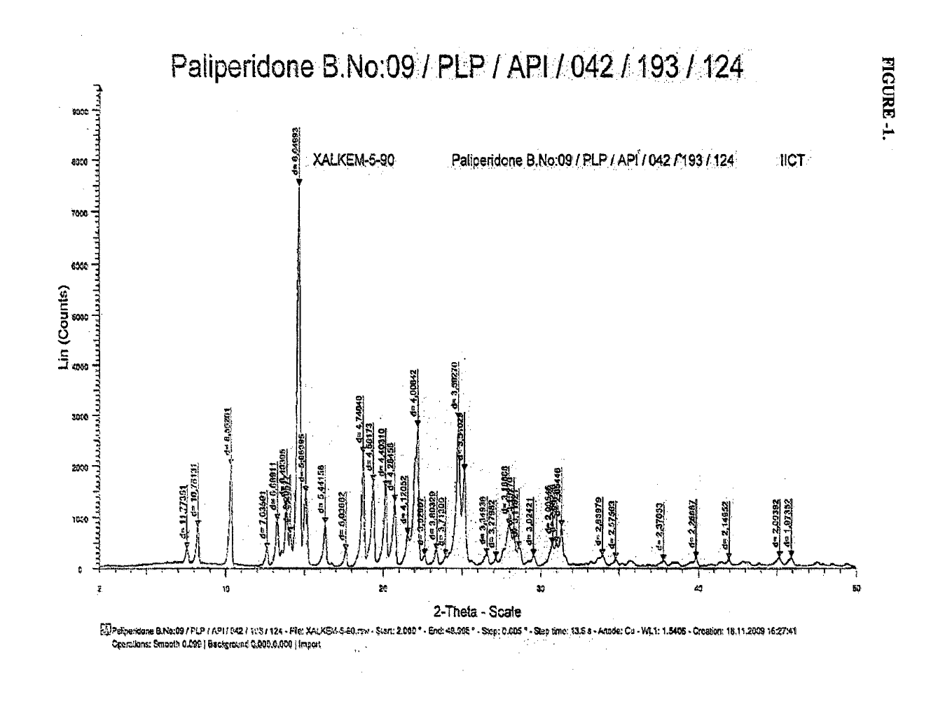

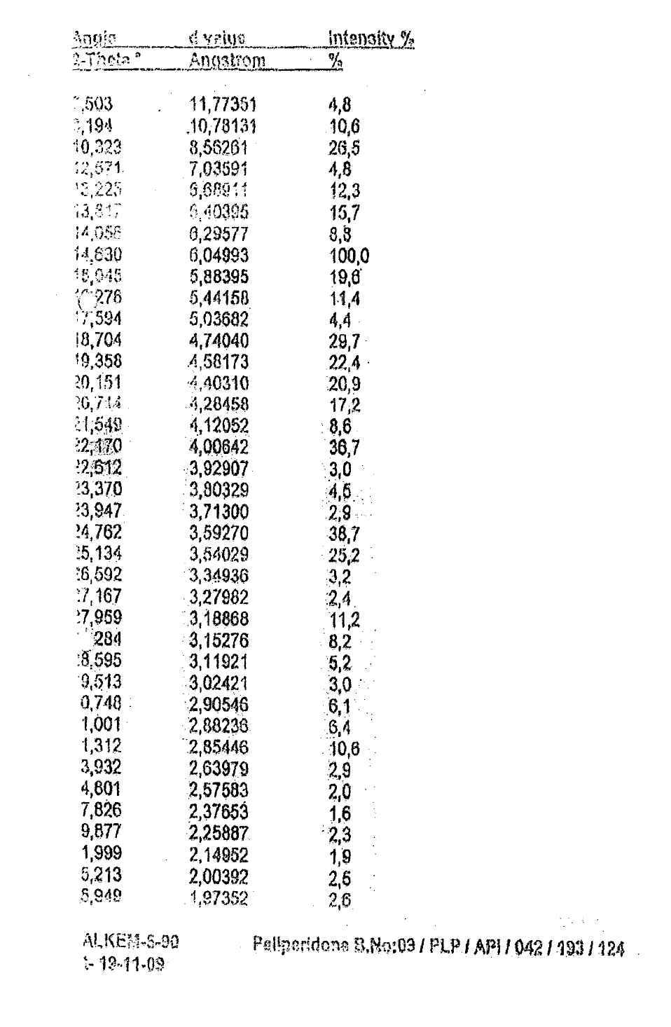

Paliperidone of purity about 98% was obtained by subjecting the various 9-O-Acylated derivatives of Paliperidone as prepared in Examples-15, 16, 17 and 18 to hydrolysis using Methanolic Ammonia or Methanolic Monomethylamine as illustrated in the examples—4 and 13 respectively. The XRPD values of the Paliperidone obtained were consistent with that of Crystalline Paliperidone Form II as depicted in FIG. 1 of this application.

1. A process for the preparation of palipieridone comprising hydrolysis of 9-O-acylated Paliperidone of Formula 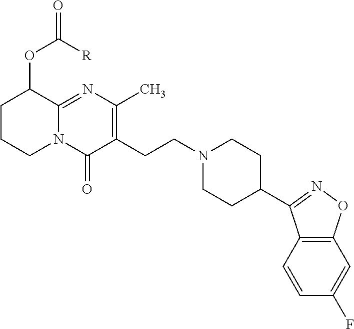
wherein R=C₁-C₁₀ straight or branched alkyl, alkyl substituted by aryl or aryl which may be unsubstituted or substituted.

2. The process according to claim 1 wherein the Paliperidone obtained is Form II.

3. The process according to claim 1 where the Paliperidone has a purity of about 98% or more.

4. The process according to claim 1 comprising hydrolysis of 9-O acetyl paliperidone of Formula 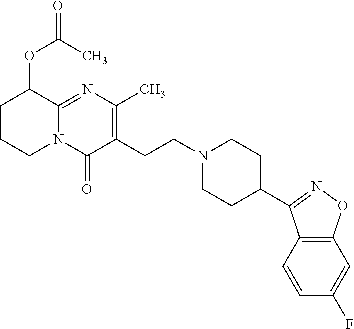
to afford Paliperidone Form II of purity of about 98% or more.

5. The process according to claim 1 wherein the hydrolysis is acidic or basic.

6. The process according to claim 5 wherein the base used for hydrolysis is selected from inorganic or organic bases selected from the group comprising sodium carbonate, potassium carbonate, sodium hydroxide, potassium hydroxide, ammonia, sodium methoxide, sodium ethoxide, potassium t-butoxide, triethyl amine, diisopropylamine, methyl amine and the like.

7. The process according to claim 6 where the base is preferably selected from Ammonia or Methylamine.

8. The process according to claim 1 comprising
a) Conversion of Crude Paliperidone to 9-O-acylated paliperidone and optionally purifying it.
b) Hydrolysis of 9-O-Acylated paliperidone.
